(19) United States
(12) Patent Application Publication
(10) Pub. No.: US 2026/0324954 A1
(43) Pub. Date: Oct. 22, 2026

(54) DEVICES FOR ACCESSING THE BILIARY TRACT

(71) Applicants: Boston Scientific Scimed, Inc., Maple Grove, MN (US); Boston Scientific Medical Device Limited, Galway (IE)

(72) Inventors: Adrián Navarro Orozco, San Jose (CR); Jose Roberto Soto Guzman, Alajuela (CR); Lucia Perez Saborio, Heredia (CR); Diego Alvarez Thuel, Alajuela (CR); Valeria Urena Quiros, San Jose (CR); Emanuel Chaves Pizarro, Cartago (CR); Raquel Carvajal Alvarado, San Jose (CR); Josué Rivera Araya, San Jose (CR); Fernando Valerio Guerrero, Alajuela (CR); Vivian Hernandez, Heredia (CR); William Bertolino, Framingham, MA (US); Kate Beatrice Concannon, Brighton, MA (US); Jonathan Melchor, Providence, RI (US); Julia Grace Pasco-Anderson, Somerville, MA (US); Jennifer Evelyn Edmondson, North Grafton, MA (US); Arun Adhikarath Balan, Pune (IN); Pooja Bhuvanesh Kulkarni, Pune (IN); Harinandan H. Nair, Ernakulam (IN); Shrikant Vasant Raut, Pune (IN); Deepak Kumar Sharma, Gurgaon (IN); Samriddhi Jain, Pune (IN); Mihir Satish Sukhatme, Mumbai (IN); Harshit Singh Chauhan, Satna (IN); Shazia Ansari, Pune (IN); Sucheta Mishra, Pune (IN); Hitendra Purohit, Pune (IN); Sumedh Deshpande, Pune (IN); Yogesh Kishor Vikharankar, Pune (IN)

(73) Assignees: Boston Scientific Scimed, Inc., Maple Grove, MN (US); Boston Scientific Medical Device Limited, Galway (IE)

(21) Appl. No.: 19/649,617

(22) Filed: Apr. 16, 2026

Related U.S. Application Data
(60) Provisional application No. 63/790,270, filed on Apr. 17, 2025.

(51) Int. Cl. A61B 1/005; A61B 1/00

(52) U.S. Cl. CPC A61B 1/0052; A61B 1/0008; A61B 1/00131

(57) ABSTRACT

Medical devices and accessories for accessing the biliary tract include a tubular member with a tapered distal region adapted for endoscope insertion. The distal region can expand to accommodate endoscope advancement and contract upon retraction, utilizing accordion folds or axial slots that splay outward. An accessory device includes a mounting member with tapered prongs that spread apart as an endoscope advances. The devices may include proximal handles with steering and locking mechanisms.

### CROSS-REFERENCE TO RELATED APPLICATIONS

This application claims the benefit of U.S. Provisional Patent Application Ser. No. 63/790,270 filed on Apr. 17, 2025, the disclosure of which is incorporated herein by reference.

### TECHNICAL FIELD

The present disclosure is directed to medical devices. More particularly, the present disclosure relates to medical devices for accessing the biliary tract

### BACKGROUND

A number of medical procedures involve accessing the biliary tract, including the cystic duct and the common bile duct. An example includes the LTCBDE (Laparoscopic Transcystic Common Bile Duct Exploration), which may be used in treating stones within the common bile duct. In some cases, the common bile duct may be accessed from within the cystic duct in order to minimize risks of bile leakage and/or stricture. The LTCBDE procedure may include advancing a small endoscope known as a choledochoscope into the cystic duct.

### SUMMARY

This disclosure provides design, material, method, and use alternatives for medical devices, including but not limited to medical devices for accessing the biliary tract. An example may be found in a medical device that is adapted for accessing an interior of a biliary tract. The medical device includes a tubular member that extends from a proximal region to a distal region and defines a lumen extending therethrough. The distal region is adapted for insertion through an opening formed within the biliary tract. The distal region tapers to a distal end of the distal region and is the distal region adapted to accommodate an endoscope extending therethrough.

Alternatively or additionally to any example above, the tubular member may include a flexible sheath.

Alternatively or additionally to any example above, the distal region may be adapted to expand when an endoscope is advanced distally through the distal portion and to contract when the endoscope is retracted proximally.

Alternatively or additionally to any example above, the distal region may include a plurality of axially extending slots dividing the distal region into segments that splay radially outwardly from a tapered configuration when the endoscope is advanced distally.

Alternatively or additionally to any example above, the distal region may taper to the distal end via a plurality of accordion folds within the distal region.

Alternatively or additionally to any example above, the medical device may further include a shape retainer that is secured to the distal end and that is biased to a position in which the shape retainer urges the distal region into the plurality of accordion folds.

Alternatively or additionally to any example above, the medical device may further include a proximal handle that is disposed within the proximal region.

Alternatively or additionally to any example above, the medical device may further include a reversible locking mechanism that is disposed within the distal region and is actuatable from the proximal handle.

Alternatively or additionally to any example above, the medical device may further include a steering mechanism that is actuatable from the proximal handle.

Another example may be found in a medical device that is adapted for accessing an interior of a cystic duct. The medical device includes an elongate shaft that extends from a proximal region to a distal region and defines a lumen extending therethrough. The distal region is adapted for insertion through an opening formed within the cystic duct and tapers to a distal end of the distal region. The distal region is adapted to accommodate an endoscope extending therethrough.

Alternatively or additionally to any example above, the distal region may be adapted to expand when an endoscope is advanced distally through the distal portion and to contract when the endoscope is retracted proximally.

Alternatively or additionally to any example above, the distal region may include a plurality of axially extending slots dividing the distal region into segments that splay radially outwardly from a tapered configuration when the endoscope is advanced distally.

Alternatively or additionally to any example above, the medical device may further include a shape retainer that is secured to the distal end and is biased to a position in which the shape retainer urges the distal region into a plurality of accordion folds.

Alternatively or additionally to any example above, the medical device may further include a reversible locking mechanism disposed within the distal region.

Alternatively or additionally to any example above, the medical device may further include a steering mechanism.

Another example may be found in an accessory device for assisting entry of an endoscope through a tubular member and into an interior of a biliary tract. The tubular member has a distal region. The accessory device includes a mounting member that is dimensioned for a frictional fit with the distal region of the tubular member and a plurality of prongs that are coupled to the mounting member. Each of the plurality of prongs have an initial tapered configuration in which each of the plurality of prongs taper to a common point, and advancing an endoscope distally through the medical device pushes each of the plurality of prongs away from the initial tapered configuration.

Alternatively or additionally to any example above, the mounting member may include an annular ring.

Alternatively or additionally to any example above, the plurality of prongs may include angled prongs that are rotatably secured relative to the annular ring.

Alternatively or additionally to any example above, the plurality of prongs may include thin wires that are secured to the annular ring.

Alternatively or additionally to any example above, the tubular member may include a sheath.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE DRAWINGS

The drawings illustrate the design and utility of configurations of the present disclosure. It should be noted that the figures are not drawn to scale and that elements of similar structures or functions are represented by like reference numerals throughout the figures. The drawings are not to be considered as limiting the scope of the disclosure. The present disclosure is described and explained with additional specificity and detail through the use of the accompanying drawings, in which:

FIG. 1A is a schematic view of a proximal portion of an illustrative medical device for accessing an interior of the biliary tract;

FIG. 1B is a schematic view of a distal portion of the illustrative medical device of FIG. 1A;

FIG. 1C is a cross-sectional view taken along the line 1C-1C of FIG. 1B;

FIGS. 2, 3, and 4 are schematic views showing a process of accessing an interior of the cystic duct using the illustrative medical device of FIG. 1A;

FIG. 5 is a schematic view of an illustrative medical device for accessing an interior of the biliary tract;

FIG. 6 is an enlarged view of a portion of the illustrative medical device of FIG. 5;

FIG. 7 is a schematic view of the illustrative medical device of FIG. 5, shown with an endoscope extending distally from the illustrative medical device;

FIG. 8 is a schematic view of an illustrative medical device for accessing an interior of the biliary tract;

FIG. 9 is a view of a distal region of the illustrative medical device of FIG. 8

FIG. 10 is an enlarged view of the distal region shown in FIG. 9, showing inclusion of a shape retainer;

FIG. 11 is a view of the distal region of the illustrative medical device of FIG. 8, showing the distal region expanded by an endoscope being advanced distally therethrough;

FIG. 12 is a schematic view of an illustrative obturator usable with the illustrative medical device of FIG. 8;

FIG. 13 is a cross-sectional view taken along the line 13-13 of FIG. 12;

FIGS. 14A, 14B, and 14C are schematic views showing a process of accessing an interior of the cystic duct using the illustrative medical device of FIG. 8;

FIG. 15 is a schematic view of an illustrative medical device for accessing an interior of the biliary tract;

FIG. 15A is a cross-sectional view taken along the line 15A-15A of FIG. 15;

FIG. 16 is a schematic view of a handle portion of the illustrative medical device of FIG. 15;

FIG. 17A is a schematic view of an illustrative locking mechanism forming a part of the illustrative medical device of FIG. 15, with the illustrative locking mechanism shown in an unactuated configuration;

FIG. 17B is a schematic view of the illustrative locking mechanism of FIG. 17A, with the illustrative locking mechanism shown in an actuated configuration;

FIG. 18 is a schematic view of a tunnel instrument used with a trocar for accessing an interior of the cystic duct;

FIGS. 19A, 19B, and 19C are schematic views of illustrative tunnel instruments that may be used for accessing the interior of the cystic duct;

FIG. 20 is a schematic view of an illustrative medical device that may be used to access the interior of the cystic duct;

FIG. 21A is a schematic view of an illustrative steerable medical device that may be used to access the interior of the cystic duct;

FIG. 21B is an enlarged view of a distal portion of the illustrative steerable medical device of FIG. 21A;

FIG. 21C is an enlarged view of a proximal portion of the illustrative steerable medical device of FIG. 21A;

FIGS. 22A and 22B are schematic views of an illustrative accessory device coupled to a tunneling instrument, showing use of the illustrative accessory device in accessing an interior of the cystic duct; and

FIGS. 23A, 23B, and 23C are schematic views of an illustrative accessory device coupled to a tunneling instrument, showing use of the illustrative accessory device in accessing an interior of the cystic duct.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the disclosure.

A variety of medical procedures involve accessing an interior of a patient's biliary tract. The biliary tract includes several components that work together to produce, store, and release bile for digesting fat in the small intestine. The biliary tract includes the liver, the gallbladder and the bile ducts. The liver produces bile, which is a fluid that helps digest fat. The gallbladder stores and concentrates bile and releases bile into the small intestine when needed. The bile ducts include intrahepatic bile ducts that start in the liver and merges into larger ducts, eventually forming the left and right hepatic ducts. The bile ducts include extrahepatic bile ducts including the common hepatic duct (formed by the left and right hepatic ducts), the cystic duct (from the gallbladder), and the common bile duct, which is formed by the union of the common hepatic duct and the cystic duct. In some cases, the biliary tract may be accessed through an incision formed within the cystic duct. As an example, a procedure known as a LTCBDE (Laparoscopic Transcystic Common Bile Duct Exploration), may involve accessing an interior of the cystic duct. The LTCBDE procedure may be used in treating stones within the common bile duct, and involves advancing a small endoscope known as a choledochoscope into an opening formed in the cystic duct and then advancing the endoscope through the cystic duct.

There can be difficulties in gaining access to the cystic duct due to the potentially difficult angles in which the choledochoscope approaches an incision made in the cystic duct. There can also be difficulties in accessing and advancing within the cystic duct due to the internal structure such as the valves of Heister (spiral mucosal folds) present within the cystic duct.

In some instances, a medical device is adapted for accessing an interior of a biliary tract such as accessing a cystic duct. The medical device includes a tubular member that extends from a proximal region to a distal region and that defines a lumen extending therethrough. The distal region is adapted for insertion through an opening formed within the biliary tract and tapers to a distal end of the distal region. The distal region is adapted to accommodate an endoscope extending therethrough.

In some cases, the tubular member may be a flexible sheath. In some cases, the tubular member may be a rigid trocar. The distal region of the tubular member may be adapted to expand when an endoscope is advanced distally through the distal portion and the distal region of the tubular member may be adapted to contract when the endoscope is retracted proximally. In some cases, the distal region may include a plurality of axially extending slots that divide the distal region into segments that splay radially outwardly from a tapered configuration when the endoscope is advanced distally.

In some cases, the distal region may taper to the distal end via a plurality of accordion folds within the distal region. The medical device may further include a shape retainer that is secured to the distal end and that is biased to a position in which the shape retainer urges the distal region into the plurality of accordion folds.

In some cases, the medical device may further include a proximal handle that is disposed within the proximal region. In some cases, the medical device may further include a reversible locking mechanism that is disposed within the distal region and that is actuatable from the proximal handle. In some cases, the medical device may further include a steering mechanism that is actuatable from the proximal handle.

In some instances, a medical device is adapted for accessing an interior of a cystic duct. The medical device includes an elongate shaft that extends from a proximal region to a distal region and defines a lumen extending therethrough. The distal region is adapted for insertion through an opening formed within the cystic duct and tapers to a distal end of the distal region. The distal region is adapted to accommodate an endoscope extending therethrough.

In some cases, the distal region may be adapted to expand when an endoscope i advanced distally through the distal portion and to contract when the endoscope is retracted proximally. In some cases, the distal region may include a plurality of axially extending slots dividing the distal region into segments that splay radially outwardly from a tapered configuration when the endoscope is advanced distally. In some cases, the medical device may further include a shape retainer that is secured to the distal end and that is biased to a position in which the shape retainer urges the distal region into a plurality of accordion folds. In some cases, the medical device may further include a reversible locking mechanism disposed within the distal region. In some cases, the medical device may further include a steering mechanism.

In some instances, an accessory device may be adapted for assisting entry of an endoscope through a tubular member and into an interior of a biliary tract. The tubular member has a distal region. The accessory device includes a mounting member that is dimensioned for a frictional fit with the distal region of the tubular member and a plurality of prongs that are coupled to the mounting member. Each of the plurality of prongs have an initial tapered configuration in which each of the plurality of prongs taper to a common point. Advancing an endoscope distally through the medical device pushes each of the plurality of prongs away from the initial tapered configuration.

In some cases, the mounting member may include an annular ring. The plurality of prongs may include angled prongs that are rotatably secured relative to the annular ring. In some cases, the plurality of prongs may include thin wires that are secured to the annular ring. The tubular member may be a sheath. In some cases, the tubular member may be a trocar.

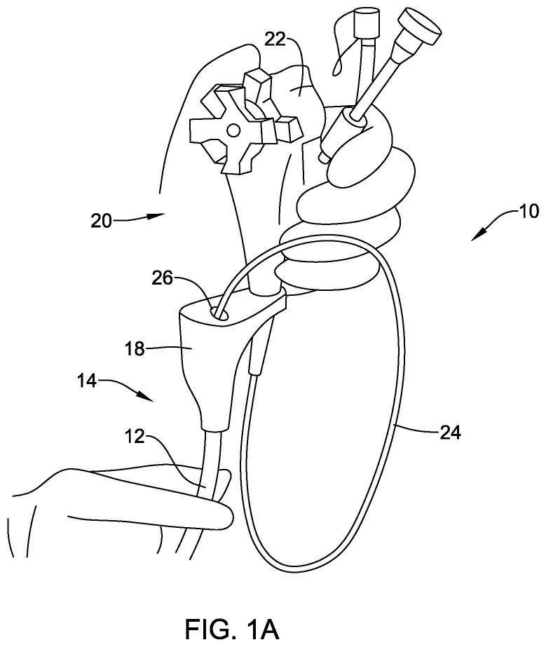

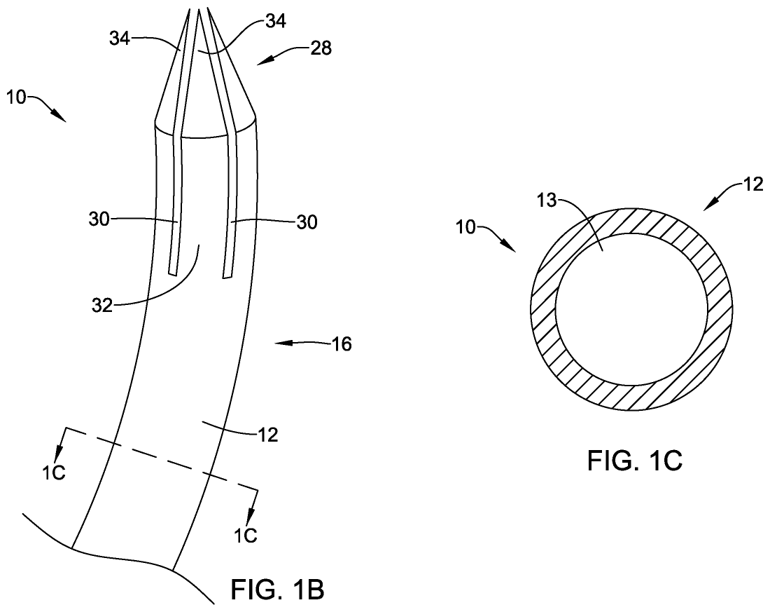

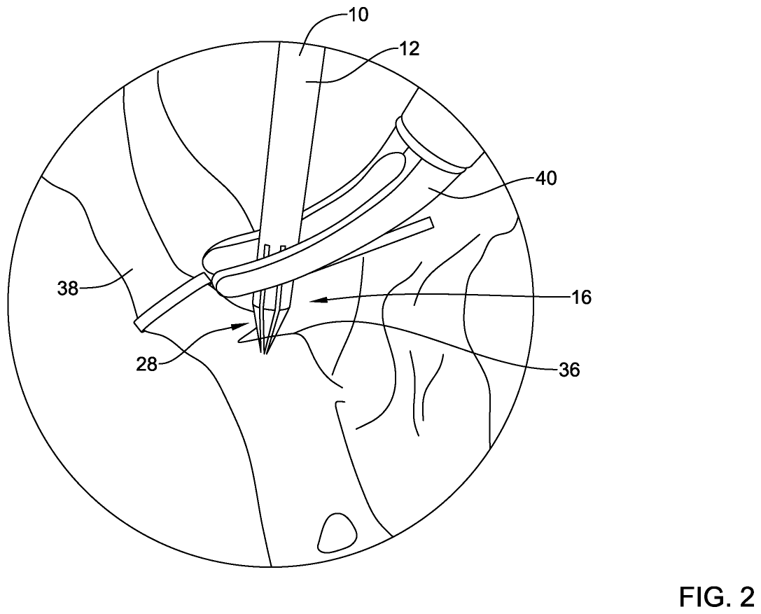

FIG. 1A is a schematic view of a proximal portion of an illustrative medical device 10 for accessing an interior of the biliary tract and FIG. 1B is a schematic view of a distal portion of the illustrative medical device 10. FIG. 1C is a cross-sectional view taken along the line 1C-1C of FIG. 1B.

The medical device 10 may include an elongate shaft 12 that extends from a proximal region 14 to a distal region 16. The elongate shaft 12 may be considered as being an example of a tubular member. The elongate shaft 12 may have a simple single layer structure as shown in FIG. 1C, defining a lumen 13 that extends through the elongate shaft 12. In some cases, the elongate shaft 12 may include two or more different polymeric layers, and may in some instances include one or more reinforcing layers such as a woven, knitted, or braided reinforcing layer. In some cases, the elongate shaft 12 may have varying degrees of flexibility. For example, a distal portion of the elongate shaft 12 may be more flexible than a proximal portion of the elongate shaft 12 in order to facilitate inserting the distal portion of the elongate shaft 12 into the biliary tract. The proximal portion of the elongate shaft 12 may be stiffer than the distal portion of the elongate shaft 12 for better column support and pushability. In some cases, the elongate shaft 12 may include a PTFE (polytetrafluoroethylene) coating to reduce frictional forces. The medical device 10 includes a proximal hub 18. In some cases, the proximal hub 18 may be adapted to be releasably secured to a proximal portion of an endoscope 20. As an example, the endoscope 20 may be a small endoscope known as a choledochoscope. An example choledochoscope is the Discovery Spyglass®, available commercially from Boston Scientific of Marlborough, MA, USA. The endoscope 20 includes a proximal handle 22 and an elongate shaft 24 that extends from the proximal handle 22. In some cases, as shown, the proximal hub 18 of the medical device 10 may form a compressive or snap-fit connection with the proximal handle 22 of the endoscope 20 and the elongate shaft 24 may extend into and through an aperture 26 formed within the proximal hub 18.

The distal region 16 of the elongate shaft 12 may include a tapered portion 28. In some cases, the distal region 16 may include a number of axially-extending slots 30. Adjacent axially-extending slots 30 define corresponding segments 32. In some cases, the corresponding segments 32, which in some cases may be referred to as being petal-like segments 32, may have an initial configuration (as shown in FIG. 1B) in which the segments 32 have distal ends 34 that are positioned adjacent to each other, thereby helping to form the tapered portion 28. The narrow distal end of the tapered portion 28 helps in advancing the elongate shaft 12 into and through an incision (or ductotomy) formed within a desired location of the patient's biliary tract. In some cases, an incision (or ductotomy) may be formed within the patient's cystic duct, which is part of their biliary tract. Once the distal region 16 of the elongate shaft 12 has been sufficiently advanced into the cystic duct, another medical device such as the endoscope 20 may be advanced through the elongate shaft 12. As the endoscope 20 passes through the distal region 16, the endoscope 20 may cause the segments 32 to splay in an outward direction (as seen for example in FIGS. 3 and 4), thereby allow the endoscope 20 to pass through the distal region 16 and extend distally out of the elongate shaft 12.

While only two axially-extending slots 30 are visible in FIG. 1B, the distal region 16 of the elongate shaft 12 may include any number of axially-extending slots 30, defining any number of segments 32 therebetween. In some cases, at least some of the axially-extending slots 30 may extend proximally from a distal end of the distal region 16 (denoted for example by the distal ends 34 of each of the segments 32). In some cases, at least some of the axially-extending slots 30 may extend proximally from the distal end of the region 16 and may extend proximally beyond the tapered portion 28 of the distal region 16. In some cases, the distal region 16 of the elongate shaft 12 may be formed of a flexible material such as a polymer. In some cases, the distal region 16 may be adapted to default to the initial configuration shown in FIG. 1B, to splay outwardly when a medical device such as the endoscope 20 is advanced therethrough, and may return to that initial configuration when the medical device such as the endoscope 20 is withdrawn proximally and is no longer extending through the distal region 16. In some examples, the distal region 16 may be biased to the initial configuration shown in FIG. 1B.

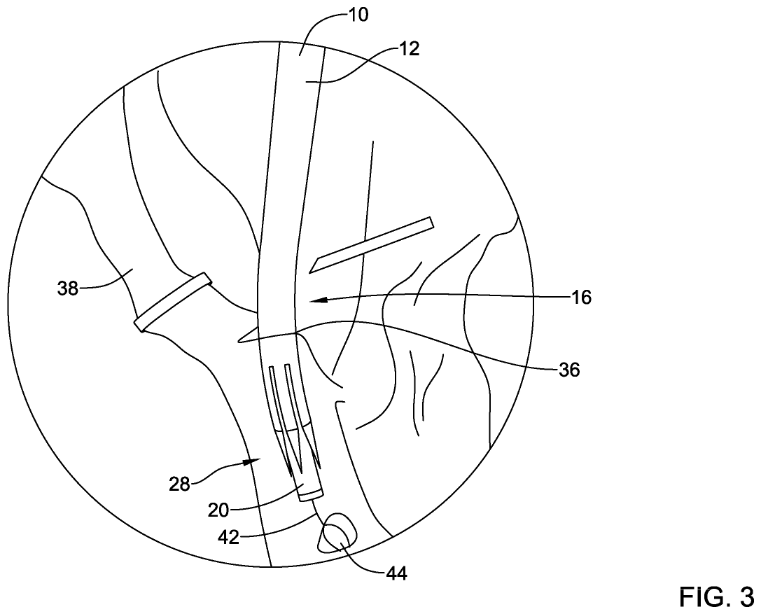

An illustrative use of the medical device 10 is shown in FIGS. 2, 3, and 4. The elongate shaft 24 of the endoscope 20 may be advanced through the elongate shaft 12 of the medical device 10. In some cases, the endoscope 20 may be advanced through the distal region 16 of the elongate shaft 12 such that a camera that forms part of the endoscope 20 may be used for visualization. Once the endoscope 20 nears a ductotomy (incision) 36 that has been formed within a cystic duct 38 (FIG. 2), the endoscope 20 may be withdrawn proximally back into the elongate shaft 12, allowing the tapered portion 28 to revert to its initial tapered configuration. In some cases, a grasper 40 may be used to help guide the distal region 16 of the elongate shaft 12 into the ductotomy 36. In some cases, the grasper 40 may not be used. As shown, the tapered portion 28 is seen as extending through the ductotomy 36.

Once the medical device 10 has entered into the cystic duct 38, the endoscope 20 may be advanced distally through the distal region 16 of the elongate shaft 12. This can be seen in FIG. 3, in which the endoscope 20 extends distally from the elongate shaft 12, with the distal region 16 and/or the tapered portion 28 in an expanded flared out configuration that allows the endoscope 20 to pass therethrough. At this point, a user may utilize a basket device 42 to snare a stone 44. Once the stone 44 has been snared by the basket device 42, the medical device 10 and the endoscope 20 may be withdrawn proximally, as shown in FIG. 4, to withdraw the stone 44. Although snaring a stone is depicted in FIGS. 3 and 4, other suitable medical procedures may be performed.

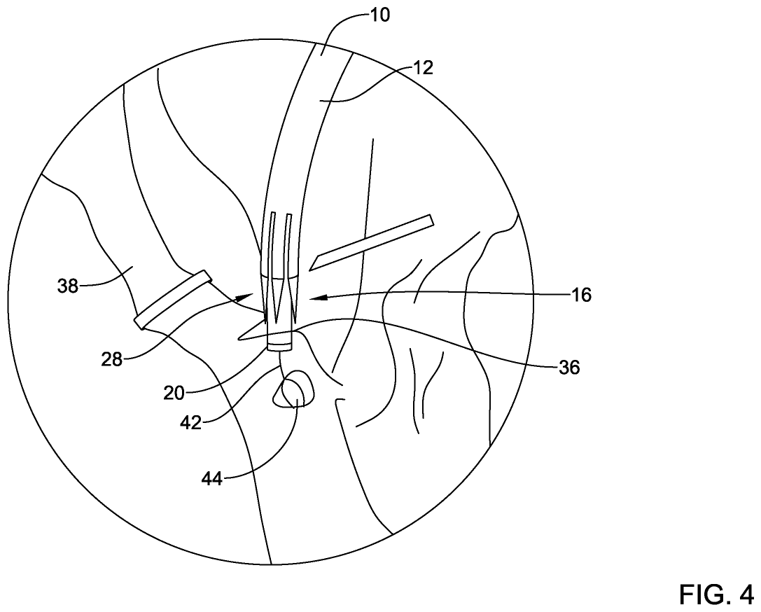

FIG. 5 is a schematic view of an illustrative medical device 46 that may be used for accessing an interior of the biliary tract, such as the cystic duct 38 (FIGS. 2-4). The illustrative medical device 46 may include an elongate shaft 48 that extends from a proximal region 50 to a distal region 52. The distal region 52 of the elongate shaft 48 may include a tapered region 54 that tapers to a distal end 56. The tapered region 54 may include axially-aligned slots 58. The tapered region 54 may allow for easier access through the ductotomy 36 (FIGS. 2-4), and may be atraumatic.

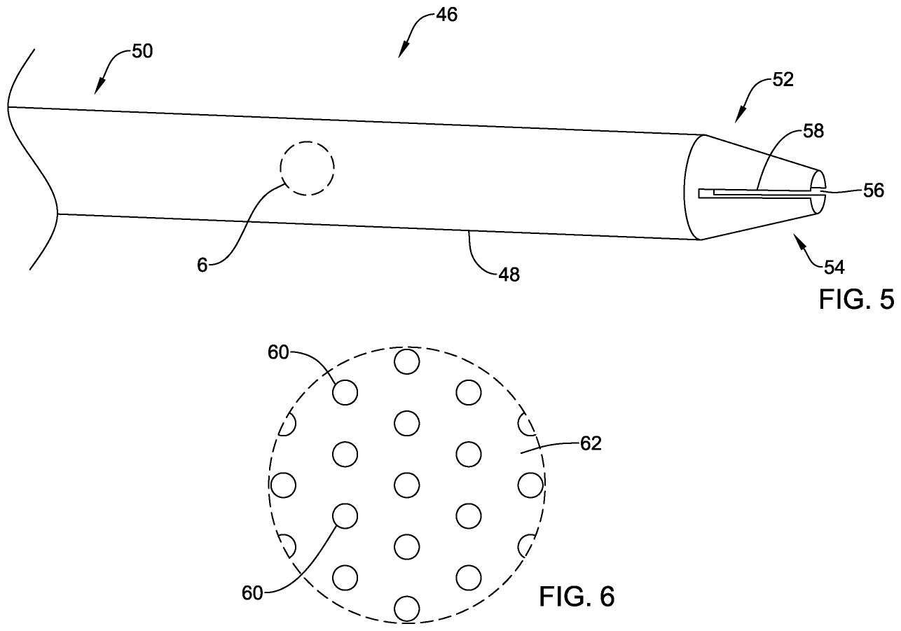

The elongate shaft 48 may include features 60 disposed on an outer surface 62 of the elongate shaft 48. The features 60 can help to provide the outer surface of the elongate shaft 48 with a roughened surface that helps the elongate shaft 48 to engage the interior of the cystic duct 38 (FIGS. 2-4). The features 60, which are schematically shown in FIG. 6, may be bumps or raised portions on the outer surface 62 of the elongate shaft 48. In some cases, the features 60 may be added to the outer surface 62 of the elongate shaft 48. In some cases, the features 60 may be formed on the outer surface 62 of the elongate shaft 48 by removing material from the outer surface 62, such as via an abrasive process.

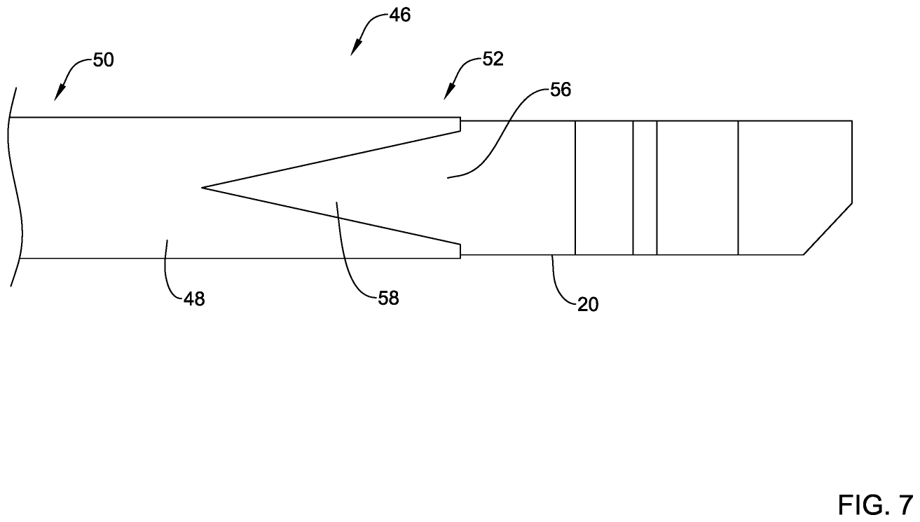

As shown in FIG. 7, the endoscope 20 has been advanced distally through the distal region 52 of the medical device 46. As a result, the slots 58 may be expanded, opening up to allow the endoscope 20 to pass through and out of the distal region 52 of the medical device 46. Later, once the endoscope 20 is withdrawn proximally, the tapered region 54 may revert to its tapered configuration. In some examples, the tapered region 54 may be biased to the tapered configuration.

The elongate shaft 48 may be placed over the endoscope 20 prior to use. The elongate shaft 48, which may be polymeric, may have a length that is sufficient to allow a physician or other user to be able to separately manipulate the medical device 46 and the endoscope 20 so that they are able to translate one of the medical device 46 and the endoscope 20 relative to the other of the medical device 46 and the endoscope 20. In use, the medical device 46 and the endoscope 20 (together as an assembly) may be advanced over a guidewire (not shown) and into the cystic duct 38 (FIGS. 2-4). As a result of the features 60, the elongate shaft 46 may begin to grab onto the cystic duct 38. The user may continue to push on the endoscope 20, which may cause the endoscope 20 to translate relative to the medical device 46 and the endoscope 20 may advance distally through the distal region 52 and/or the tapered region 54.

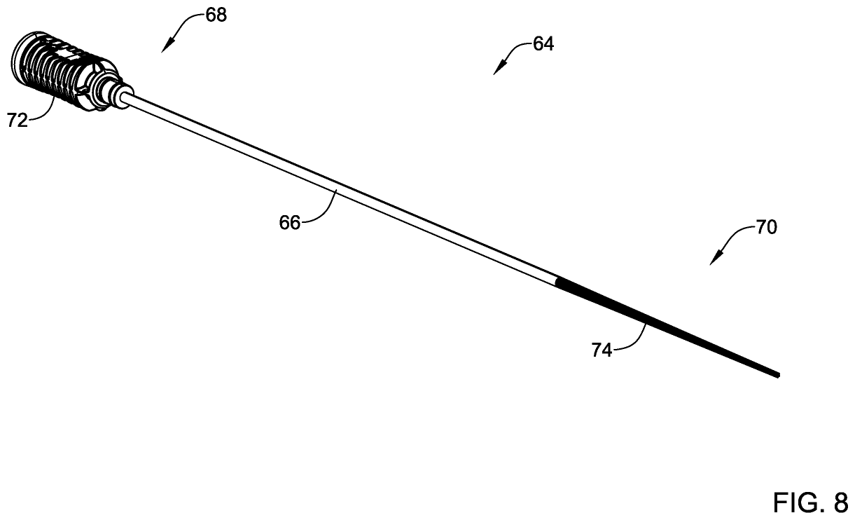

FIG. 8 is a schematic view of an illustrative medical device 64 for accessing an interior of the biliary tract, such as the cystic duct 38 (FIGS. 2-4). The illustrative medical device 64 may include an elongate shaft 66 that extends from a proximal region 68 to a distal region 70. The elongate shaft 66 may be adapted to accommodate a guidewire extending through the medical device 64. In some cases, the elongate shaft 66 may have varying degrees of flexibility. For example, a distal portion of the elongate shaft 66 may be more flexible than a proximal portion of the elongate shaft 66 in order to facilitate inserting the distal portion of the elongate shaft 66 into the biliary tract. The proximal portion of the elongate shaft 66 may be stiffer than the distal portion of the elongate shaft 66 for better column support and pushability. In some cases, the medical device 64 may include a proximal hub 72 that is secured to the proximal region 68 of the elongate shaft 66. In some cases, the elongate shaft 66 may be a multilayered polymeric tube that includes one or more reinforcing braids disposed on or within the multilayered polymeric tube. The distal region 70 of the elongate shaft 66 may include a flexible tapered region 74. As an example, the elongate shaft 66 (proximal of the flexible tapered region 74) may have a length that ranges from 25 to 30 centimeters while the flexible tapered region 74 may have a length that ranges from 10 to 20 centimeters. Other dimensions are also contemplated.

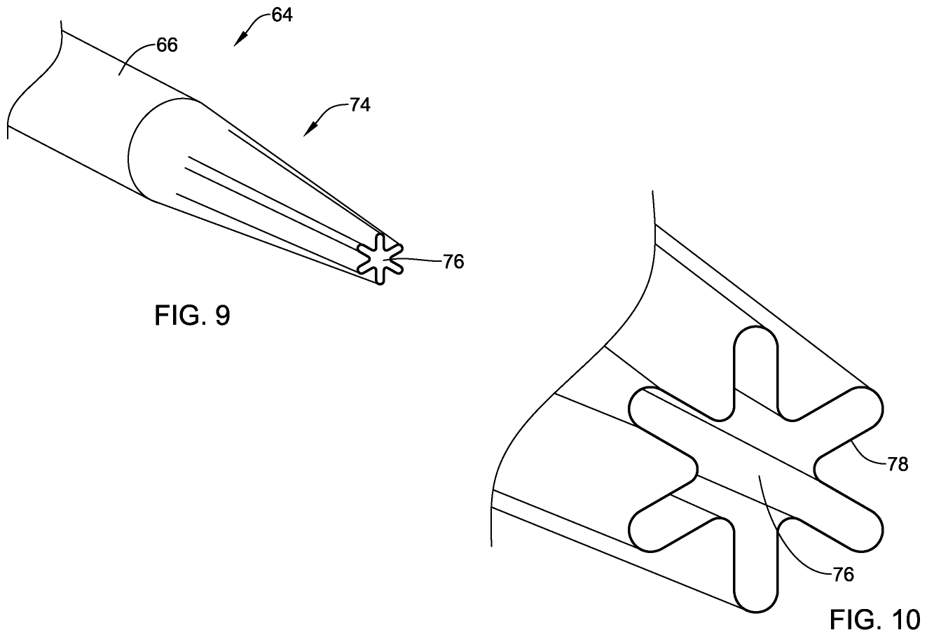

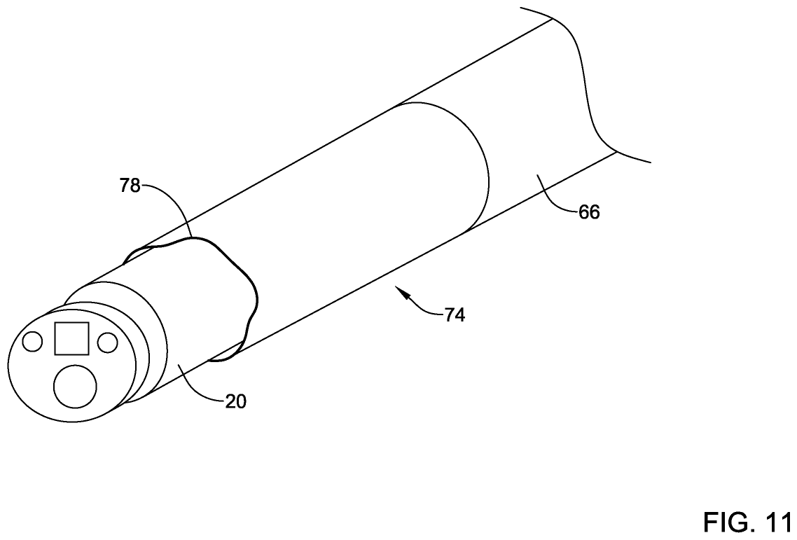

FIGS. 9 and 10 show additional details of the flexible tapered region 74. The flexible tapered region 74 may be formed of a soft, flexible polymeric material. As shown in FIG. 9, the flexible tapered region 74 may have a tapered shape as a result of the flexible tapered region 74 being folded into a number of accordion-style folds that provide the flexible tapered region 74 with a distal end 76 having a minimal diameter for better accessing an interior of the cystic duct 38 (FIGS. 2-4) via a ductotomy 36 (FIGS. 2-4). In some cases, the flexible tapered region 74 may include a shape retainer 78 that is secured to the polymer forming the flexible tapered region 74. In some cases, the shape retainer 78 may be formed of a shape memory material such as a shape memory polymer or a shape memory metal. As an example, the shape retainer 78 may be formed of nitinol. The compressed configuration shown in FIGS. 9 and 10 may represent a remembered shape to which the shape retainer 78 will return when not otherwise constrained or deformed from its remembered shape. In some examples, the shape retainer 78 may be biased to the remembered shape.

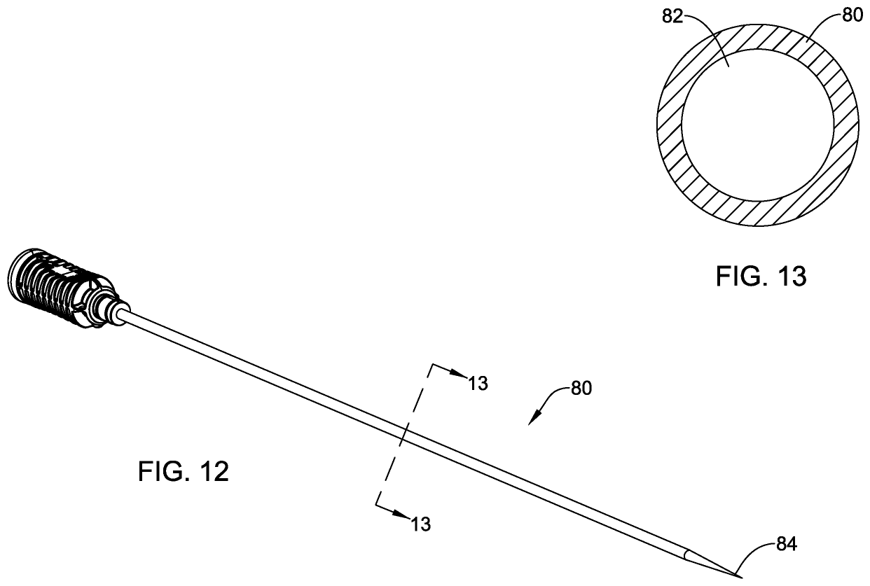

FIG. 11 shows the shape retainer 78 (and hence the flexible tapered region 74) expanded from its remembered shape by virtue of the endoscope 20 extending therethrough. When the endoscope 20 is withdrawn proximally, the shape retainer 78 will revert to its remembered shape as shown in FIGS. 9 and 10. When the shape retainer 78 reverts to its remembered shape, the soft polymer forming the rest of the flexible tapered region 74 will also revert to its tapered configuration.

In some cases, the shape retainer 78 may have any of a variety of different remembered shapes, as long as the particular remembered shape allows the flexible tapered region 74 to fold into a tapered configuration. In some cases, the flexible tapered region 74 may be joined to the rest of the elongate shaft 66 via a variety of different methods. Examples of suitable methods for joining the flexible tapered region 74 to the elongate shaft 66 include using a heat shrink tube, adhesives, and over-molding. In some cases, the medical device 64 may include features that allow for manual control of when the flexible tapered region 74 is in its tapered configuration and when the flexible tapered region 74 expands to allow other medical devices such as the endoscope 20 or the obturator 80 to be advanced through the flexible tapered region 74.

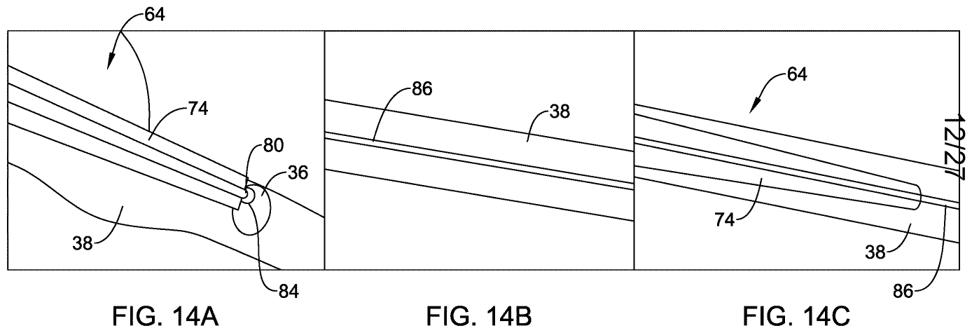

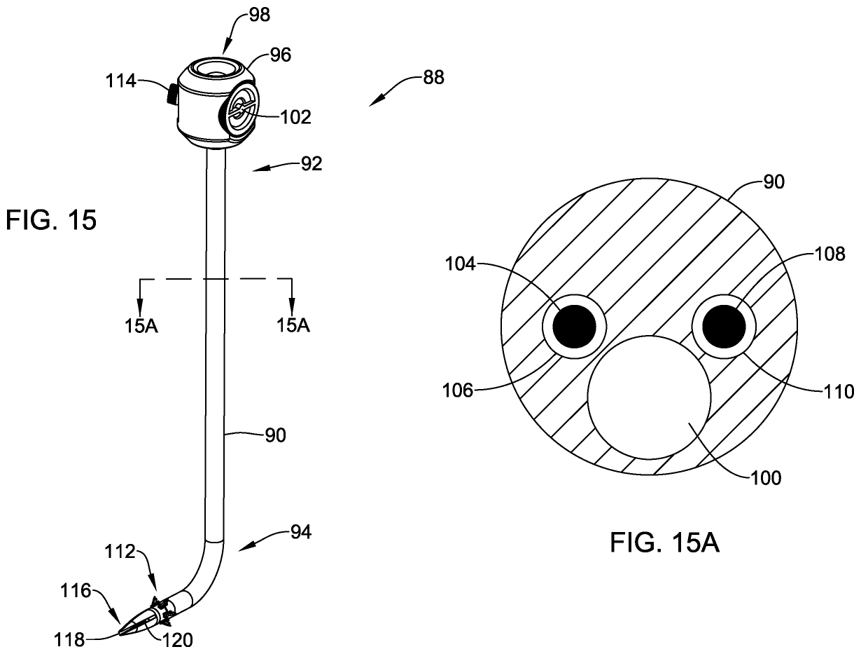

In some cases, the medical device 64 may be used in combination with an obturator 80, as shown in FIGS. 12 and 13. FIG. 12 is a schematic view of the obturator 80 and FIG. 13 is a cross-sectional view thereof, taken along the line 13-13 of FIG. 12.

As can be seen from FIGS. 12 and 13, the obturator 80 may define a lumen 82 through which other devices such as a guidewire may be advanced. The obturator 80 may include a tapered distal tip 84. In some cases, the obturator 80 may have varying degrees of flexibility. For example, a distal portion of the obturator 80 may be more flexible than a proximal portion of the elongate shaft 12 in order to facilitate inserting the distal portion of the obturator 80 into the biliary tract. The proximal portion of the obturator 80 may be stiffer than the distal region of the obturator 80 for better column support and pushability.

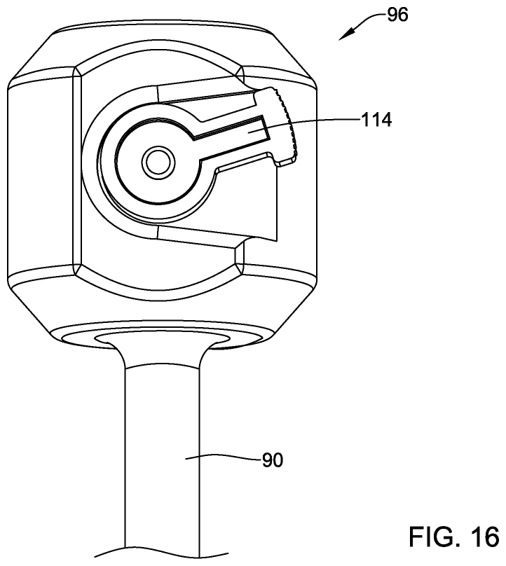

FIGS. 14A, 14B, and 14C together show an illustrative process for accessing an interior of the cystic duct 38 through the ductotomy 36. As shown in FIG. 14A, the medical device 64, with the obturator 80 extending through the medical device 64, is advanced near the ductotomy 36 formed within the cystic duct 38. In some cases, the tapered distal tip 84 helps to steer the medical device 64 through the ductotomy 36. In some cases, the obturator 80 may provide a passage (via the lumen 82) for a guidewire 86. The guidewire 86 is advanced through the cystic duct 38, as shown in FIG. 14B. Once the guidewire 86 has been advanced, the medical device 64 is advanced along the guidewire 86, within the cystic duct 38, as shown in FIG. 14C.

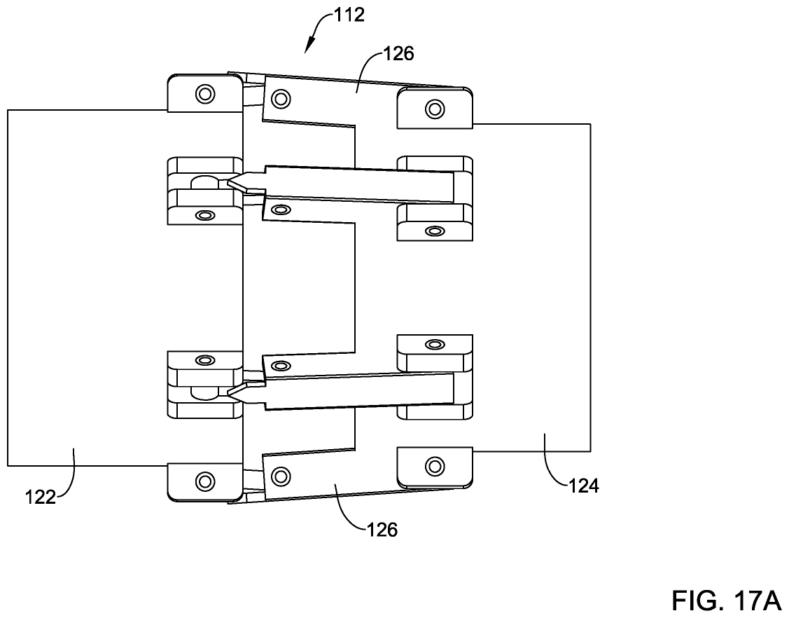

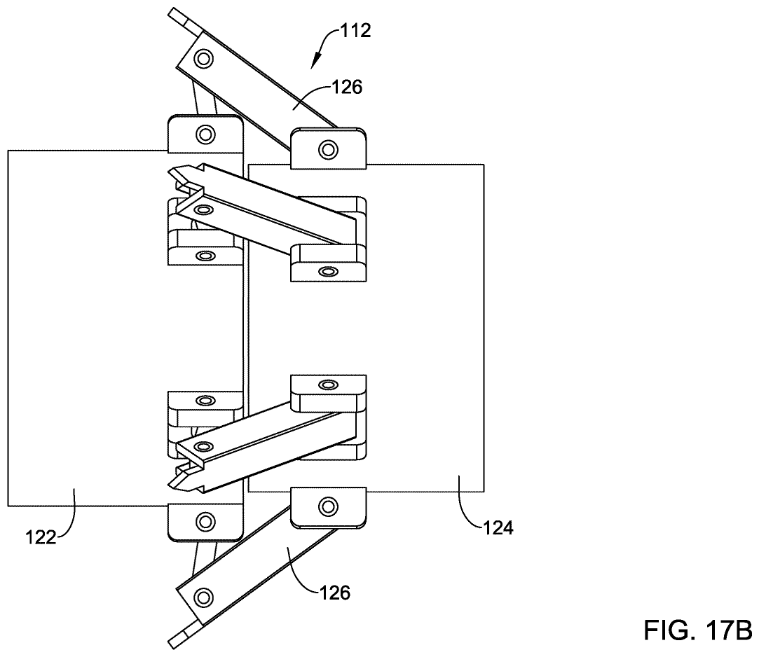

FIG. 15 is a schematic view of an illustrative medical device 88 that may be used to access the interior of the cystic duct 38 (FIGS. 2-4). FIG. 15A is a cross-sectional view taken along the line 15A-15A of FIG. 15.

The medical device 88 may be used to access the interior of the cystic duct 38 and provide a path therethrough for the endoscope 20 to access the interior of the cystic duct 38. The medical device 88 may include an elongate shaft 90 that extends from a proximal region 92 to a distal region 94. In some cases, a handle 96 may be coupled to the proximal region 92 of the elongate shaft 90. The handle 96 may include an opening 98 that leads to a lumen 100 that extends through the elongate shaft 90.

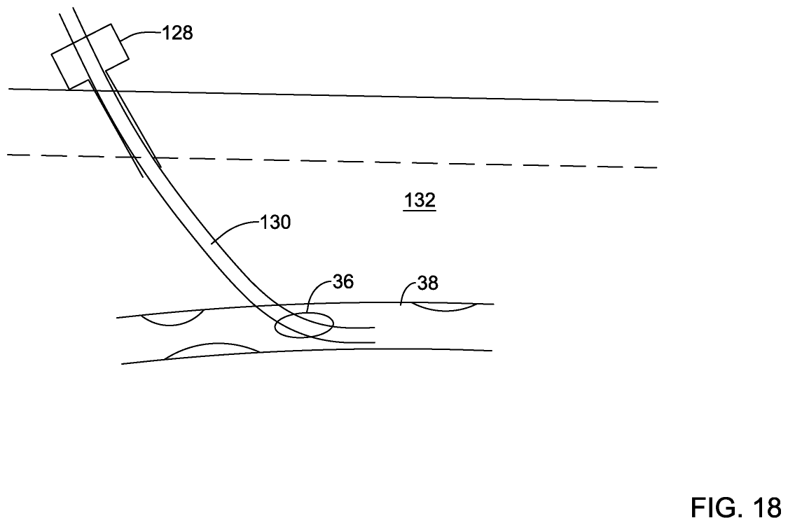

In some cases, the distal region 94 may be steerable. The handle 96 may include a knob 102 that may be operably coupled with a steering wire 104 that passes through a steering lumen 106. Rotating the knob 102 may cause the steering wire 104 to pull on the distal region 94, causing the distal region 94 to bend or deflect. In some cases, the steering mechanism may be considered as being actuatable from the handle 96. In some cases, the medical device 88 may include a reversible locking mechanism 112 that is secured to the elongate shaft 90 within the distal region 94. The handle 96 may include a lever 114 that may be used for actuating the reversible locking mechanism 112. FIG. 16 shows an opposing side of the handle 96, better illustrating the lever 114. The lever 114 may be operably coupled with a locking wire 108 that passes through a locking lumen 110. In some cases, the reversible locking mechanism 112 may be considered as being actuatable from the handle 96. In some cases, the entire medical device 88 may be rotated by rotating the handle 96, thereby also rotating the relative position of the distal region 94.

With reference to FIG. 15, the distal region 94 may include a tapered portion 116 that tapers to a minimum diameter at a distal end 118. The tapered portion 116 may include axially-extending slots 120 that allow the tapered portion 116 to constrict down to a tapered configuration as shown. When another medical device such as the endoscope 20 is advanced through the distal region 94, the tapered portion 116 may be able to expand radially outwardly to allow the endoscope 20 to advance through the distal region 94.

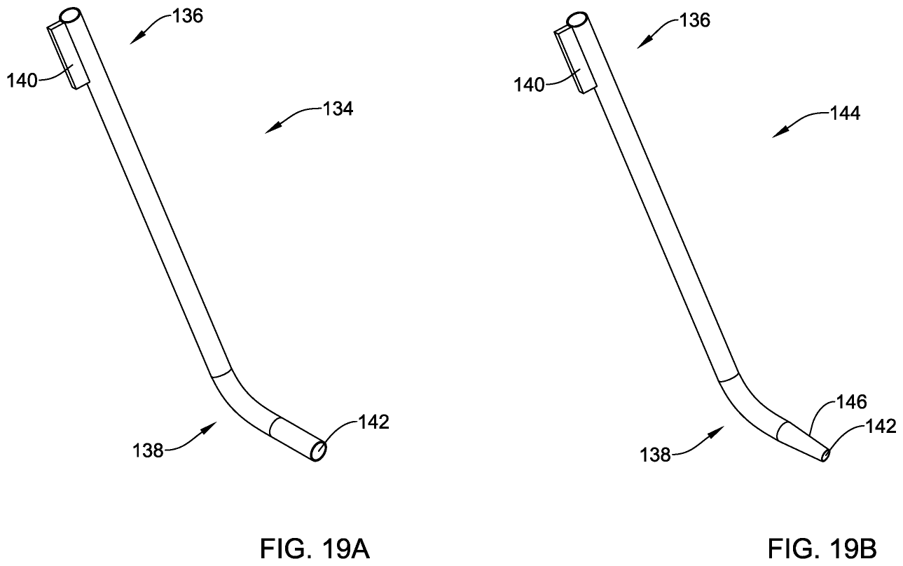

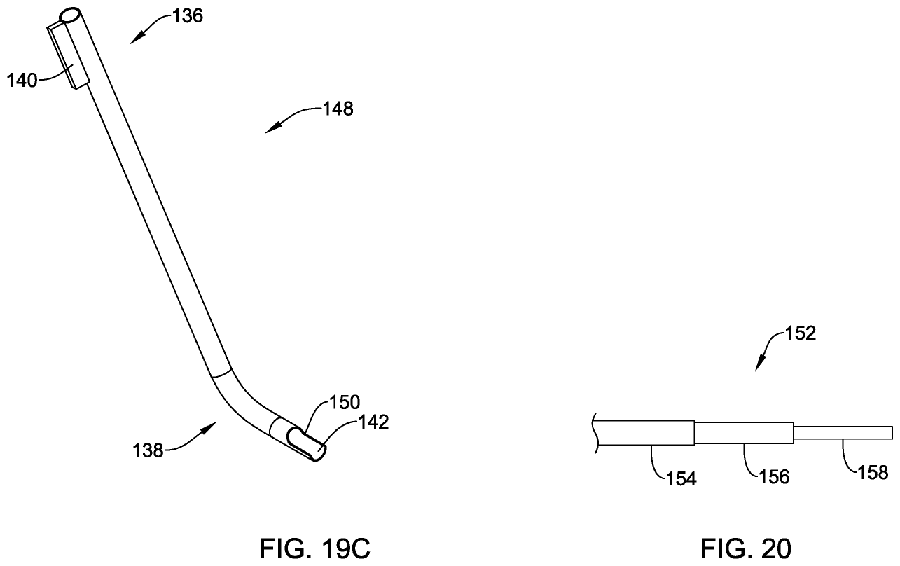

The reversible locking mechanism 112 is shown in greater detail in FIGS. 17A and 17B. In FIG. 17A, the reversible locking mechanism 112 is shown in an unactuated configuration. In FIG. 17B, the reversible locking mechanism 112 is shown in an actuated configuration in which the reversible locking mechanism 112 is adapted to interact with the interior of the cystic duct 38 (FIGS. 2-4) and help to anchor the medical device 88 in position.

The reversible locking mechanism 112 may include a fixed ring 122 that may for example be secured to the elongate shaft 90 and a moving ring 124 that may for example be secured to the distal region 94. It will be appreciated that in the unactuated configuration, the moving ring 124 is axially spaced from the fixed ring 122, and that in the actuated configuration, the moving ring 124 is closer to the fixed ring 122. Actuating the lever 114 may cause the moving ring 124 to move towards the fixed ring 122, thereby causing a number of locking levers 126 to pivot in a radially outward direction. As the locking levers 126 pivot in a radially outward direction, the locking levers 126 engage tissue within the cystic duct 38.

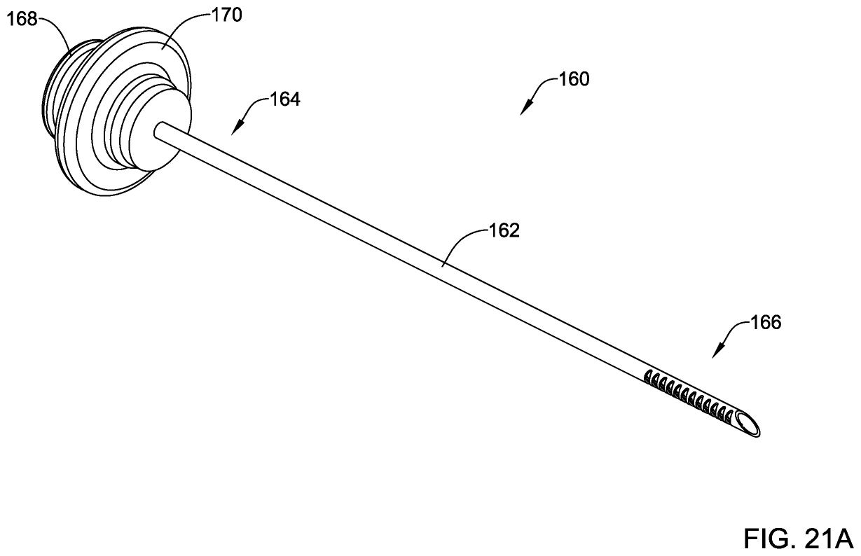

In some cases, a trocar or access point may be used to gain access through a patient's abdominal wall. In some cases, a tunnel instrument may be used to extend from the trocar or access point to a desired treatment site within the patient. FIG. 18 schematically shows a trocar 128 and a tunnel instrument 130 that may be used to extend from the trocar 128, through the abdominal cavity 132 and through the ductotomy 36 into the cystic duct 38.

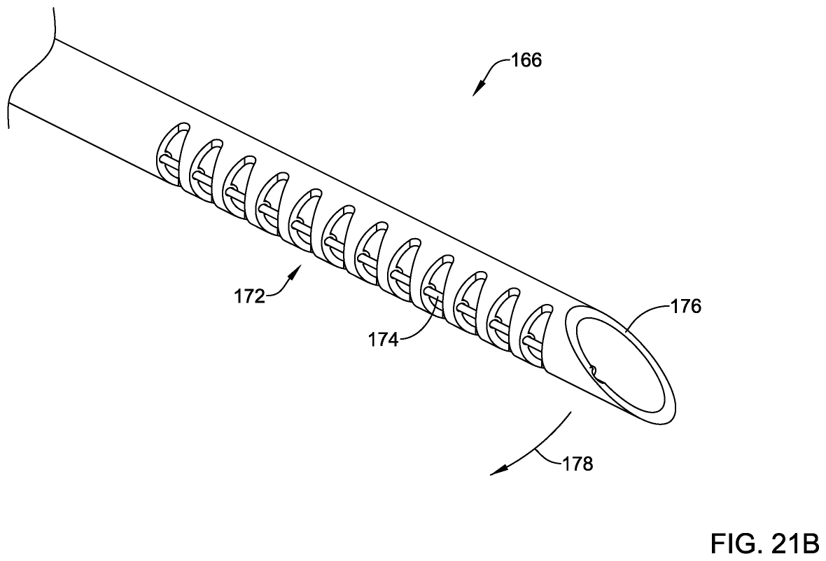

FIGS. 19A, 19B, and 19C provide examples of tunnel instruments that may be used as the tunnel instrument 130. The tunnel instruments 130, 134, 144, and 148 depicted in FIGS. 18-91C may be formed of a flexible polymer. Polyether block amide (PEBA), such as Pebax, is an example of one of the many polymers that may be used.

FIG. 19A is a schematic view of an illustrative tunnel instrument 134 that extends from a proximal region 136 to a distal region 138. In some cases, a guidewire may be used with the tunnel instrument 134. In some cases, the proximal region 136 may include a coupling feature 140 that may be used by the physician to manipulate the tunnel instrument 134 during insertion. In some cases, the coupling feature 140, or another portion of the proximal region 136, may be used to secure the tunnel instrument 134 relative to the trocar 128 (FIG. 18). The distal region 138 may be curved to better align a distal end 142 of the distal region 138 within the cystic duct 38.

FIG. 19B is a schematic view of an illustrative tunnel instrument 144. The tunnel instrument 144 may be similar to the tunnel instrument 134, but the distal region 138 of the tunnel instrument 144 has a tapered portion 146 that tapers to its distal end 142. In some cases, the tapered portion 146 may be easier to advance through the ductotomy 36 and into the cystic duct 38.

FIG. 19C is a schematic view of an illustrative tunnel instrument 148 that is similar to the tunnel instruments 134 and 144, but the distal region 138 of the tunnel instrument 148 includes an open side 150. In some cases, the open side 150 may facilitate entry through the ductotomy 36 and into the cystic duct 38.

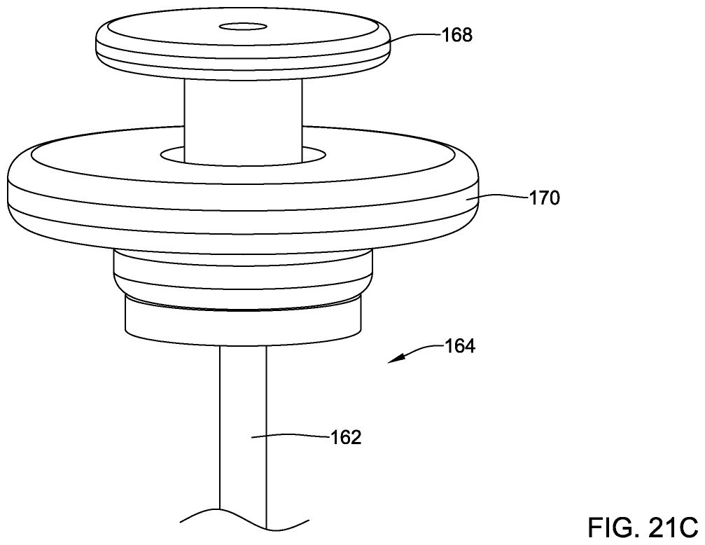

In some cases, a tunnel instrument may not have a fixed length, but may instead include several stacked conical tubes that go from smallest diameter at a proximal end to a largest diameter at a distal end. FIG. 20 is a schematic view of an illustrative tunnel instrument 152 that includes a first conical tube 154, a second conical tube 156, and a third conical tube 158. Inserting a medical device such as the endoscope 20 causes the tunnel instrument 152 to naturally being to open or extend. In some cases, the tunnel instrument 152 may include detent features (not shown) that limit the axial elongation of the tunnel instrument 152.

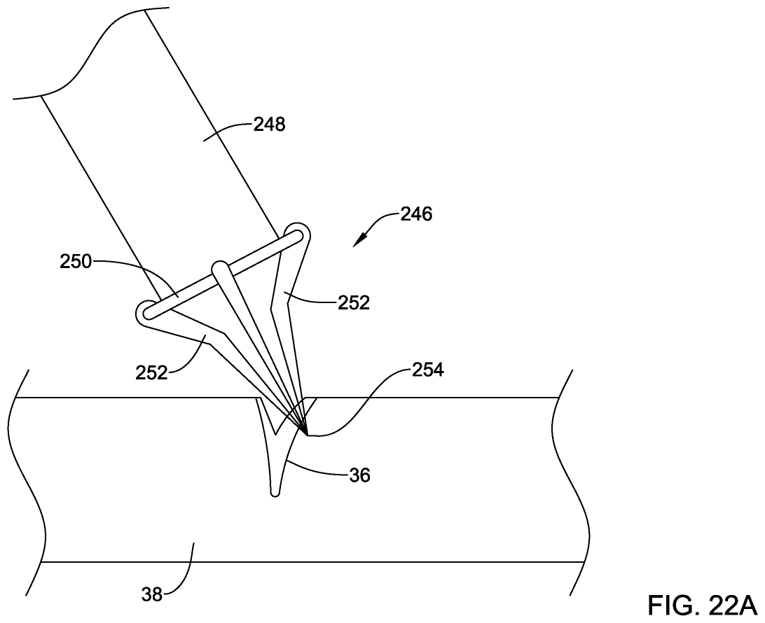

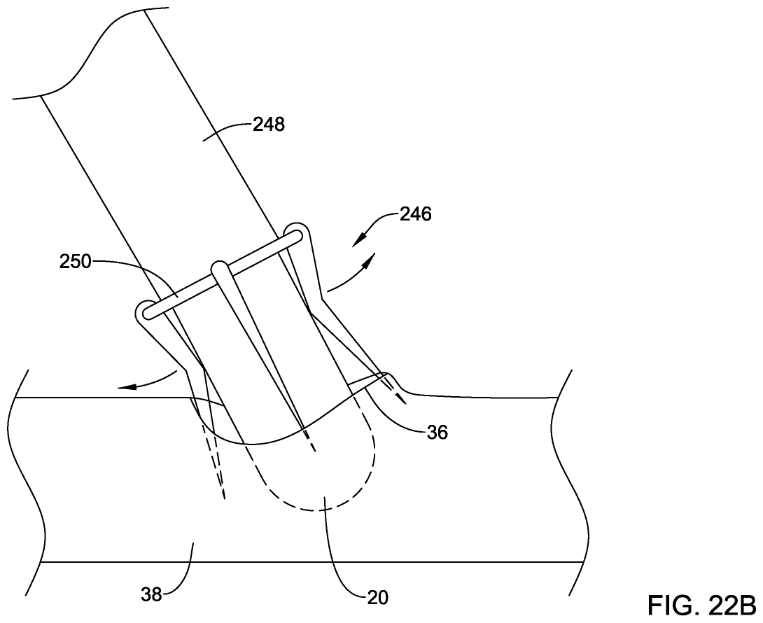

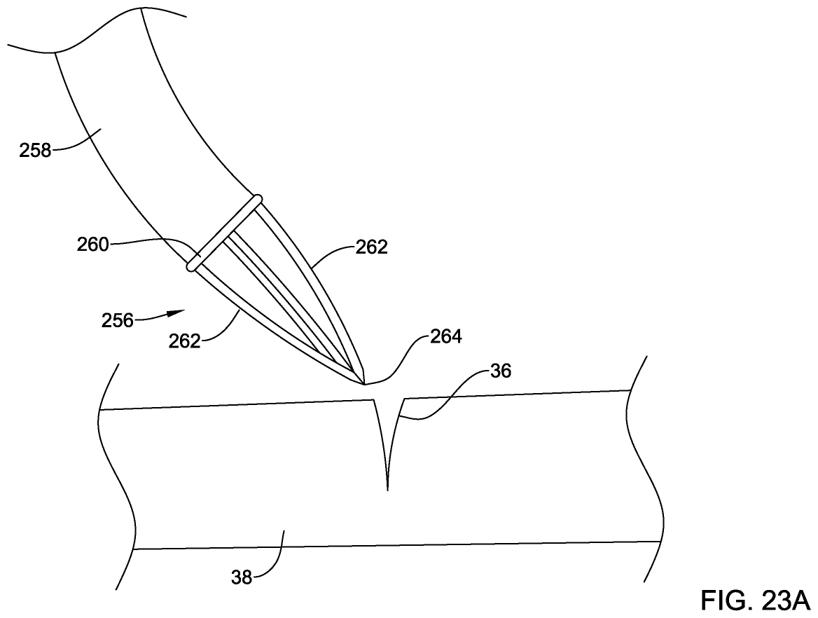

FIG. 21A is a schematic view of a steerable tunnel instrument 160. FIGS. 21B and 21C provide enlarged views of portions of the steerable tunnel instrument 160. The steerable tunnel instrument 160 includes an elongate shaft 162 that extends from a proximal region 164 to a distal region 166. The proximal region 164 includes a fixed handle 168 and a moving handle 170 that is adapted to translate relative to the fixed handle 168. The distal region 166 includes a plurality of slots 172 that may be cut into one side of the distal region 166 in order to provide the distal region 166 with preferential bending. A pull wire 174 extends from the moving handle 170 to a distal end 176 of the distal region 166. Moving the moving handle 170 towards the fixed handle 168 effectively shortens a length of the pull wire 174, causing the distal region 166 to bend in a direction indicated by an arrow 178.

In some cases, an accessory device that is configured for facilitating cystic duct access may be coupled to a tubular member in order to help advance an endoscope through the tubular member and into the cystic duct 38. As an example, an accessory device may include a mounting member that is dimensioned for a frictional fit with the distal region of the tubular member and a plurality of prongs that are coupled to the mounting member. Each of the plurality of prongs may have an initial tapered configuration in which each of the plurality of prongs taper to a common point. Advancing an endoscope distally through the medical device may push each of the plurality of prongs away from the initial tapered configuration.

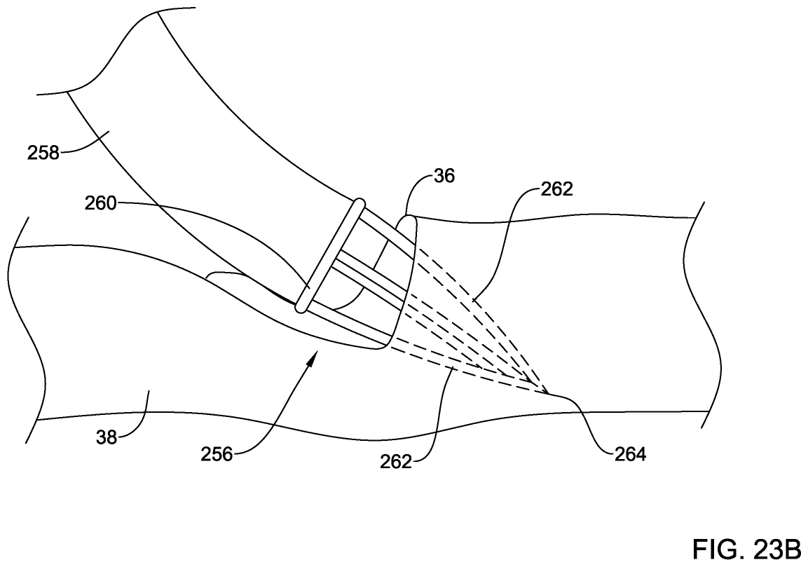

FIGS. 22A and 22B are schematic views of an illustrative accessory device 246 that is shown mounted onto a rigid trocar 248. FIG. 22A shows the accessory device 246 in a collapsed configuration and FIG. 22B shows the accessory device 246 in an expanded configuration.

The accessory device 246 depicted in FIGS. 22A and 22B may include an annular ring 250 that forms a compressive fit with the rigid trocar 248. The accessory device 246 may include a number of angled prongs 252 that are rotatably or pivotably secured to the annular ring 250. The accessory device 246 may include any number of angled prongs 252. In some cases, the accessory device 246 may include four angled prongs 252 that are equally spaced out circumferentially around the annular ring 250. In an initial collapsed configuration, each of the angled prongs 252 come together at a distal point 254 that is tapered for easier access through the ductotomy 36. In FIG. 22B, distal advancement of the endoscope 20 through the rigid trocar 248 has caused each of the angled prongs 252 to pivot about the annular ring 250, and move radially outwardly to allow the endoscope 20 to pass through. In some examples, the accessory device 256 may be biased to the initial collapsed configuration.

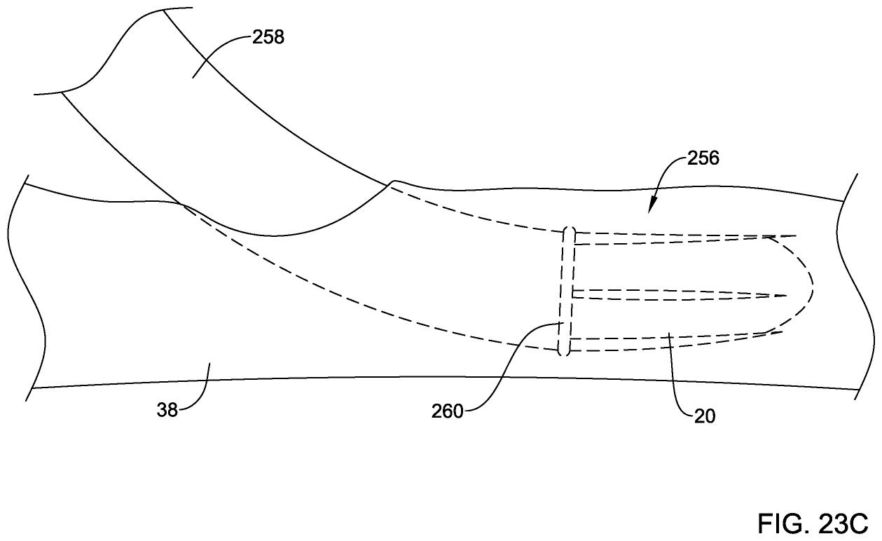

FIGS. 23A, 23B, and 23C are schematic views of an illustrative accessory device 256 that is shown mounted onto a flexible sheath 258. FIGS. 23A and 23B show the accessory device 256 in a collapsed configuration and FIG. 23C shows the accessory device 256 in an expanded configuration.

The accessory device 256 depicted in FIGS. 23A-23C may include an annular ring 260 that forms a compressive fit with the flexible sheath 258. The accessory device 256 may include a number of thin wires 262 that are secured to the annular ring 260 and that taper to a distal point 264 while in the collapsed configuration. The accessory device 256 may include any number of thin wires 262. In some cases, the accessory device 256 may include four thin wires 262 that are equally spaced out circumferentially around the annular ring 260. As shown in FIG. 23B, the accessory device 256 may be advanced through the ductotomy 36 and into an interior of the cystic duct 38 while in an initial configuration. In FIG. 23C, the endoscope 20 has been advanced through the flexible sheath 258, thereby causing the thin wires 262 to bend out of the way, allowing the endoscope 20 to pass therethrough. In some examples, the accessory device 256 may be biased to the initial configuration.

The materials that can be used for the various components of the systems presently disclosed may include those commonly associated with medical devices. The devices and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS®400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility may be imparted. For example, the devices described herein, or portions thereof, may be made of a material that does not substantially distort the image and create substantial artifacts (i.e., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A medical device adapted for accessing an interior of a biliary tract, the medical device comprising:
a tubular member extending from a proximal region to a distal region and defining a lumen extending therethrough;
the distal region adapted for insertion through an opening formed within the biliary tract, the distal region tapering to a distal end of the distal region; and
the distal region adapted to accommodate an endoscope extending therethrough.

2. The medical device of claim 1, wherein the tubular member comprises a flexible sheath.

3. The medical device of claim 1, wherein the distal region is adapted to expand when the endoscope is advanced distally through the distal region and to contract when the endoscope is retracted proximally.

4. The medical device of claim 3, wherein the distal region includes a plurality of axially extending slots dividing the distal region into segments that splay radially outwardly from a tapered configuration when the endoscope is advanced distally.

5. The medical device of claim 3, wherein the distal region tapers to the distal end via a plurality of accordion folds within the distal region.

6. The medical device of claim 5, further comprising a shape retainer secured to the distal end, the shape retainer is biased to a position in which the shape retainer urges the distal region into the plurality of accordion folds.

7. The medical device of claim 1, further comprising a proximal handle disposed within the proximal region.

8. The medical device of claim 7, further comprising a reversible locking mechanism disposed within the distal region and actuatable from the proximal handle.

9. The medical device of claim 7, further comprising a steering mechanism that is actuatable from the proximal handle.

10. A medical device adapted for accessing an interior of a cystic duct, the medical device comprising:
an elongate shaft extending from a proximal region to a distal region and defining a lumen extending therethrough;
the distal region adapted for insertion through an opening formed within the cystic duct, the distal region tapering to a distal end of the distal region; and
the distal region adapted to accommodate an endoscope extending therethrough.

11. The medical device of claim 10, wherein the distal region is adapted to expand when the endoscope is advanced distally through the distal region and to contract when the endoscope is retracted proximally.

12. The medical device of claim 11, wherein the distal region includes a plurality of axially extending slots dividing the distal region into segments that splay radially outwardly from a tapered configuration when the endoscope is advanced distally.

13. The medical device of claim 10, further comprising a shape retainer secured to the distal end, wherein the shape retainer is biased to a position in which the shape retainer urges the distal region into a plurality of accordion folds.

14. The medical device of claim 10, further comprising a reversible locking mechanism disposed within the distal region.

15. The medical device of claim 10, further comprising a steering mechanism.

16. An accessory device for assisting entry of an endoscope through a tubular member and into an interior of a biliary tract, the tubular member having a distal region, the accessory device comprising:
a mounting member that is dimensioned for a frictional fit with the distal region of the tubular member; and
a plurality of prongs coupled to the mounting member, each of the plurality of prongs in an initial tapered configuration in which each of the plurality of prongs taper to a common point; and
wherein advancing the endoscope distally through the accessory device pushes each of the plurality of prongs away from the initial tapered configuration.

17. The accessory device of claim 16, wherein the mounting member comprises an annular ring.

18. The accessory device of claim 17, wherein the plurality of prongs comprises angled prongs that are rotatably secured relative to the annular ring.

19. The accessory device of claim 17, wherein the plurality of prongs comprises thin wires that are secured to the annular ring.

20. The accessory device of claim 17, wherein the tubular member comprises a sheath.
